SEQUENCE LISTING (12 sequences)

SEQ ID NO: 1 - protein, 10 aa, Homo sapiens
  N-terminus neo-epitope biomarker of collagen type X alpha 1
GIATKGLNGP

SEQ ID NO: 2 - protein, 6 aa, Homo sapiens
  N-terminus neo-epitope biomarker of collagen type X alpha 1
GIATKG

SEQ ID NO: 3 - protein, 11 aa, Artificial Sequence
  N-terminus elongated peptide
AGIATKGLNGP

SEQ ID NO: 4 - protein, 9 aa, Artificial Sequence
  N-terminus truncated peptide
IATKGLNGP

SEQ ID NO: 5 - protein, 10 aa, Artificial Sequence
  Biotinylated peptide
  SITE 10: Biotin with optional linker between biotin and peptide
GIATKGLNGP

SEQ ID NO: 6 - protein, 13 aa, Artificial Sequence
  Immunogenic peptide
  SITE 13: Keyhole limpet hemocyanin
GIATKGLNGPGGC

SEQ ID NO: 7 - protein, 16 aa, Artificial Sequence
  Peptide fragment identified by mass spectrometry
PGSKGDPGSPGPPGPA

SEQ ID NO: 8 - protein, 14 aa, Artificial Sequence
  Peptide fragment identified by mass spectrometry
SKGDPGSPGPPGPA

SEQ ID NO: 9 - protein, 10 aa, Rattus norvegicus
  Rat alignment sequence to SEQ ID NO 1
GIVTKGLNGP

SEQ ID NO: 10 - protein, 10 aa, Mus musculus
  Mouse alignment sequence to SEQ ID NO 1
GIATKGLNGP

SEQ ID NO: 11 - protein, 10 aa, Bos taurus
  Bovine alignment sequence to SEQ ID NO 1
GIAVKGLNGP

SEQ ID NO: 12 - protein, 10 aa, Canus lupus familiaris
  Dog alignment sequence to SEQ ID NO 1
GVATKGLNGP

(19) United States
(12) Patent Application Publication
(10) Pub. No.: US 2020/0173991 A1
(43) Pub. Date: Jun. 4, 2020

(54) Collagen Type X Alpha-1 Assay

(71) Applicant: Nordic Bioscience A/S, Herlev (DK)

(72) Inventors: Yi He, Virum (DK); Anne-Cecilie Bay-Jensen, Copenhagen S (DK); Morten Karsdal, Kobenhavn Ø (DK)

(73) Assignee: Nordic Bioscience A/S, Herlev (DK)

(21) Appl. No.: 16/634,158

(22) PCT Filed: Jul. 27, 2018
(86) PCT No.: PCT/EP2018/070430

§ 371 Date: Jan. 26, 2020

(30) Foreign Application Priority Data
Jul. 27, 2017 (GB) 1712071.8

(51) Int. Cl. G01N 33/564; C07K 16/18

(52) U.S. Cl. CPC G01N 33/564; G01N 2333/78; C07K 2317/34; C07K 16/18

(57) ABSTRACT

An antibody specifically reactive with an N-terminus neo-epitope of collagen type X alpha 1 comprised in the amino acid sequence H₂N-GIATKGLNGP, and its use in an immunoassay for evaluating a disease associated with collagen type X alpha 1, such as osteoarthritis.

### TECHNICAL FIELD

The present invention relates to an antibody which specifically reacts with an N-terminus neo-epitope of collagen type X alpha 1, and its use in a method of immunoassay for detecting and quantifying collagen type X alpha 1.

### BACKGROUND ART

Osteoarthritis (OA) is a common joint disease which is characterized by cartilage damage and loss of joint function. The etiology of OA comprises multiple factors including aging, obesity, trauma and heredity [1]. The pathogenesis of OA is poorly understood due to the heterogeneity and complexity of this disease.

Remarkably, some characteristics of OA resemble chondrocyte differentiation processes during skeletal development by endochondral ossification. In healthy articular cartilage, chondrocytes resist proliferation and terminal differentiation. By contrast, chondrocytes in diseased cartilage progressively proliferate and develop hypertrophy. Moreover, vascularization and focal calcification of joint cartilage are initiated [2-5]. The molecular events regulating chondrocyte differentiation are still unknown, but chondrocyte hypertrophy-like changes in OA have attracted more attention for study [6-8].

### Type X Collagen Alpha-1

Collagen type X alpha-1 is non-fibrillar, but forms fine pericellular filaments in association with cartilage collagen. The molecule isolated from chondrocyte cultures or from cartilage is a homotrimer of 59 kDa Collagen type X alpha-1 chains, and there have been reports of a recombinant molecule of collagen type X of approximately 75 kDa [9]. Collagen type X alpha-1 shares a similar domain structure with type VIII collagen: a central triple-helical (COL1) domain of 50 kDa is flanked by N-terminal (NC2) and C-terminal (NC1) non-triple-helical domains [10]. In addition, both collagen types represent major components of hexagonal lattice structure, in which the collagen molecules link together by interactions involving the non-triple-helical end regions.

Collagen type X alpha-1 distribution is restricted to normal fetal hypertrophic cartilage in the growth zones of long bones, vertebrae and ribs, and in adult (>21 yr) thyroid cartilage, where it may provide a scaffold to prevent local collapse as the cartilage matrix is removed during endochondral ossification [11]. It is also found in bone fracture callus, in osteoarthritic cartilage and in chondrogenic neoplasms, and may be involved in cartilage mineralization.

### Osteoarthritis

OA is generally considered to be a non-inflammatory condition of the synovial joints, predominantly knee and hips. Chondrocyte hypertrophy and cartilage calcification are key pathological events in OA. Elevated expression of network-forming type X collagen is believed to be a specific signal for chondrocyte hypertrophy [12-15] therefore type X collagen can be used as a detectable marker for said disease.

There are several proteins associated with hypertrophic chondrocytes, such as collagen type X, MMP13, osteopontin, osteocalcin [16], Indian Hedgehog [17], Runx2 [18], VEGF [19], HtrA1 [20] and Transglutaminase-2 (TG-2) [21]. Collagen type X and MMP13 are among the most widely used as markers of hypertrophic chondrocytes. However, synthesis of MMP13 can be induced in chondrocytes by inflammation and mechanical stress [22-23]. Therefore, collagen type X as a hypertrophic chondrocyte specific marker can indicate a phenotype alteration of chondrocytes.

Thus, a method which accurately quantifies the amount of collagen type X or its fragments in a biological sample may allow a better understanding of collagen type X pathologies or physiological processes affecting collagen type X turnover such as OA. Evidently there is a need for such a method.

### DESCRIPTION OF THE INVENTION

The inventors have now found that cathepsin K cleaves collagen type X alpha 1 at the peptide link between ⁴⁷⁸A-G⁴⁷⁹, resulting in the formation of the N-terminus neo-epitope biomarker H₂N-⁴⁷⁹GIATKGLNGP (SEQ ID NO: 1). This neo-epitope biomarker of collagen type X alpha 1 has been shown to correlate well with osteoarthritis.

Thus, in a first aspect the present invention relates to an antibody, wherein said antibody specifically binds to an N-terminus neo-epitope of collagen type X alpha 1 comprised in the amino acid sequence H₂N-GIATKGLNGP (SEQ ID NO: 1).

Preferably, the antibody specifically binds to the N-terminus amino acid sequence H₂N-GIATKG (SEQ ID NO: 2).

Preferably, the antibody does not specifically recognise or bind an N-extended elongated version of said N-terminus amino acid sequence. In this regard “N-extended elongated version of said N-terminus amino acid sequence” means one or more amino acids extending beyond the N-terminus of the sequence H₂N-GIATKGLNGP (SEQ ID NO: 1). For example, if the N-terminal amino acid sequence H₂N-GIATKGLNGP (SEQ ID NO: 1) was elongated by an alanine residue then the corresponding “N-extended elongated version” would be H₂N-AGIATKGLNGP . . . (SEQ ID NO: 3). Similarly, it is preferable that the antibody does not specifically recognise or bind an N-truncated shortened version of said N-terminus amino acid sequence. In this regard “N-truncated shortened version of said N-terminus amino acid sequence” means one or more amino acids removed from the N-terminus of the sequence H₂N-GIATKGLNGP (SEQ ID NO: 1). For example, if the N-terminal amino acid sequence H₂N-GIATKGLNGP (SEQ ID NO: 1) was shortened by one amino acid residue then the corresponding “N-truncated shortened version” would be H₂N-IATKGLNGP . . . (SEQ ID NO: 4).

The antibody is preferably a monoclonal antibody or fragment thereof. The invention includes a cell line producing such a monoclonal antibody or fragment thereof.

In a second aspect the present invention relates to a method of immunoassay for detecting in a biological sample fragments of collagen type X alpha 1 comprising an N-terminus neo-epitope amino acid sequence H₂N-GIATKGLNGP (SEQ ID NO: 1), said method comprising contacting said biological sample comprising said N-terminus neo-epitope amino acid sequence H₂N-GIATKGLNGP (SEQ ID NO: 1) with an antibody as described above, and determining the amount of binding of said antibody.

Preferably, said method is quantitative.

Preferably, said method is used to detect and/or quantify the amount of fragments of collagen type X alpha 1 comprising the N-terminus neo-epitope amino acid sequence H₂N-GIATKGLNGP (SEQ ID NO: 1) in biofluid.

The biofluid may be a patient derived biofluid. The biofluid may be, but is not limited to, blood, urine, synovial fluid, serum, plasma, or amniotic fluid.

The method of immunoassay may be, but is not limited to, a competition assay or a sandwich assay. The method of immunoassay may be, but is not limited to, a radioimmunoassay or an enzyme-linked immunosorbent assay.

The method may further comprise correlating the quantity of fragments of collagen type X alpha 1 comprising said N-terminus neo-epitope amino acid sequence H₂N-GIATKGLNGP (SEQ ID NO: 1) determined by said method with standard disease samples of known disease severity to evaluate the severity of a disease associated with collagen type X alpha 1.

Alternatively, or in addition to, the method may further comprise comparing the quantity of said fragments of collagen type X alpha 1 comprising said N-terminus neo-epitope amino acid sequence H₂N-GIATKGLNGP (SEQ ID NO: 1) determined by said method with standard values associated with healthy subjects to evaluate the presence and/or severity of a disease associated with collagen type X alpha 1.

In that regard, said method may further comprise correlating the quantity of fragments of collagen type X alpha 1 comprising said N-terminus neo-epitope amino acid sequence H₂N-GIATKGLNGP (SEQ ID NO: 1) determined by said method with standard osteoarthritis samples of known severity.

Similarly, said method may further comprise correlating the quantity of fragments of collagen type X alpha 1 comprising said N-terminus neo-epitope amino acid sequence H₂N-GIATKGLNGP (SEQ ID NO: 1) determined by said method with standard osteoarthritis samples of known severity in subjects of known age and gender and/or standard values associated with healthy subjects to evaluate the presence and/or severity of osteoarthritis. In this regard the comparison of samples is preferably between patient derived samples, wherein the patients (from whom the samples are derived) are of the same gender and of similar age to the standard samples.

The method may further comprise quantifying the amount of fragments of collagen type X alpha 1 comprising said N-terminus neo-epitope amino acid sequence H₂N-GIATKGLNGP (SEQ ID NO: 1) in at least two samples obtained from a subject at a first time point and at least one subsequent time point, wherein a change in the quantity of said fragments from the first time point to the at least one subsequent time point is indicative of a change in the status of a disease associated with collagen type X alpha 1 from the first time point to the at least one subsequent time point.

For example, when the disease associated with collagen type X alpha 1 is osteoarthritis, an increase in the quantity of fragments of collagen type X alpha 1 comprising said N-terminus neo-epitope amino acid sequence H₂N-GIATKGLNGP (SEQ ID NO: 1) from the first time point to the at least one subsequent time point is indicative of a deterioration in osteoarthritis in a subject from the first time point to the at least one subsequent time point. Similarly, a decrease in the quantity of fragments of collagen type X alpha 1 comprising said N-terminus neo-epitope amino acid sequence H₂N-GIATKGLNGP (SEQ ID NO: 1) from the first time point to the at least one subsequent time point is indicative of an improvement in osteoarthritis in a subject from the first time point to the at least one subsequent time point.

In a final aspect, the present invention relates to an assay kit for detecting fragments of collagen type X alpha 1 comprising the N-terminus neo-epitope amino acid sequence H₂N-GIATKGLNGP (SEQ ID NO: 1) in a biological sample, said kit comprising an antibody as described above and at least one of:
a streptavidin coated 96 well plate
a biotinylated peptide H₂N-GIATKGLNGP-L-Biotin (SEQ ID NO: 5), wherein L is an optional linker
a secondary antibody for use in a sandwich immunoassay
a calibrator peptide comprising the sequence H₂N-GIATKGLNGP
an antibody biotinylation kit
an antibody HRP labeling kit
an antibody radiolabeling kit
an assay visualization kit

### Definitions

As used herein the term “N-terminus” refers to the extremity of a polypeptide, i.e. at the N-terminal end of the polypeptide, and is not to be construed as meaning in the general direction thereof.

As used herein the term “N-terminus neo-epitope” refers to an N-terminus epitope formed by cleavage of a protein by a protease. For the instant invention this means an N-terminus epitope formed by cleavage of Collagen type X alpha-1 by cathepsin K.

As used herein the term “fragments of collagen type X alpha 1 comprising an N-terminus neo-epitope amino acid sequence H₂N-GIATKGLNGP (SEQ ID NO: 1)” means peptide fragments of collagen type X alpha 1 wherein the N-terminus of the peptide fragment is the amino acid sequence H₂N-GIATKGLNGP . . . (SEQ ID NO: 1).

As used herein the term, the term “competitive ELISA” refers to a competitive enzyme-linked immunosorbent assay and is a technique known to the person skilled in the art.

As used herein the term “sandwich immunoassay” refers to the use of at least two antibodies for the detection of an antigen in a sample, and is a technique known to the person skilled in the art.

As used herein, the term “Col10neo” is used as shorthand to describe the herein disclosed specific assay for detecting and quantifying fragments of collagen type X alpha-1 comprising the N-terminus neo-epitope amino acid sequence H₂N-GIATKGLNGP (SEQ ID NO: 1).

### DESCRIPTION OF THE FIGURES

FIG. 1: Peptide-binding specificity of mAb. The reactivity of 2F4 toward a biotinylated synthetic peptide, GIATKGLNGP-k(Biotin) (SEQ ID NO: 5), was completely displaced by adding of 1000 ng/mL selection peptide. In contrast, a slight or no displacement was observed with elongation of selection peptide or truncation of selection peptide at the same concentration.

FIG. 2: in vitro cleavage of cartilage by enzymes. 2A: MMPs cleaved human cartilage. 2B: ADAMTSs cleaved human cartilage. 2C: Cathepsin K cleaved human cartilage. Data shown as mean±SD. There was no increased level of Col10/C in none of the tested MMPs or ADAMTSs solution compared to the one without adding proteases in the digestion buffer. Conversely, Cathepsin K yielded the largest amount indicating its ability of releasing the fragment carrying the neo-epitope of 479GIATKGLNGP.

FIG. 3: Immunolocalization of 479GIATKG in cartilage. 3A: normal mouse IgG. 3B: 11G8, anti-C terminus of type X collagen. 3C: 2F4, anti-479GIATKG. Type X collagen detected by 11G8 occurred in the extracellular matrix of chondrocytes in the deep zone, but it was absent from the region of calcified cartilage. C. Surprisingly, the intense staining of 479GIATKG was seen in the extracellular matrix of chondrocytes from all zones in cartilage. Scale bar=500 μM

FIG. 4: Association between KL grade and Col10neo level in the plasma of subjects in the C4Pain study. The data were shown as mean±95CI %. 4A: Plasma Col10neo levels in different K/L groups. There was a trend toward increased Col10neo levels with a greater K/L grade, but not reaching statistical significance. 4B: The distribution of subjects with a K/L 3-4 in plasma Col10neo level by tertile. The one-way ANOVA with post-hoc Tukey-Kramer test was used. Plasma Col10neo data were logarithmic transformed in all analyses. P value<0.05 was considered statistically significant.

FIG. 5: Plasma Col10neo levels in the NYUHJD Progression study. The data were shown as mean±95CI %. Plasma Col10neo was statistically higher in OA than healthy control (p=0.0002) or RA (p<0.0001). No significant difference in healthy control and RA was found. The one-way ANOVA with post-hoc Tukey-Kramer test was used. Plasma Col10/Cat data were logarithmic transformed in all analyses. P value<0.05 was considered statistically significant. One asterisk (*) if p<0.05; two (**) if p<0.01; three (***) if p<0.001 and four (****) if p<0.0001.

### EXAMPLES

The presently disclosed embodiments is described in the following Examples, which are set forth to aid in the understanding of the disclosure, and should not be construed to limit in any way the scope of the disclosure as defined in the claims which follow thereafter. The following examples are put forth so as to provide those of ordinary skill in the art with a complete disclosure and description of how to make and use the described embodiments, and are not intended to limit the scope of the present disclosure nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

In the following examples, the following materials and methods were employed.

### Materials

Unless otherwise stated, all materials used for experiments were of high quality and ordered from Sigma-Aldrich (Copenhagen, Denmark) or VWR (Rodedovre, Denmark). The synthetic peptides used for monoclonal antibody production and assay development were purchased from GenScript (USA). MMP-2 catalytic domain (cat # GO4MP02C), MMP-9 catalytic domain (cat # GO4MP09C) and MMP-13 catalytic domain (cat # G04MP13C) were bought from Gitto Biotech (Florence, Italy). Cathepsin K (cat #219461), Cathepsin B (cat #219362) and Cathepsin S (cat #219343) and ADAMTS-4 (cat # CC1028) were from Merck Millipore (Darmstadt, Germany). ADAMTS-5 (cat #2198-AD-20) was purchased from R&D system (Minnesota, USA).

### Selection of the Sequence for Immunization

The analysis for naturally occurring peptides in human urine from OA patients was carried out by LC-MS/MS. A significant amount of peptide fragments were identified in human OA urine. 17 peptide sequences were found to be unique to human type X collagen. Two among 17 sequences carrying the same free c-terminus located at amino acid (aa) position 478′ (Accession No.: Q03692; Database: UniProt) were present in the diseased urine, indicating the cleavage occurring between the bond of A⁴⁷⁸-⁴⁷⁹G. The first 10 aa, ⁴⁷⁹GIATKGLNGP⁴⁸⁸ (SEQ ID NO: 1), of the free N-terminal end generated by this cleavage was selected for immunization. The sequence alignment from different species was also analyzed using the Basic Local Alignment Search Tool (BLAST, see Table 1).

Monoclonal Antibody (mAb) Production

Six female Balb/C mice of 6-7 weeks of age were immunized subcutaneously with emulsified GIATKGLNGP-GGC-KLH (SEQ ID NO: 6) with Sigma Adjuvant System®(cat # S6322, Sigma-Aldrich). 100 μg of emulsified KLH-conjugate with adjuvant was repeatedly injected into mouse every 3^{rd} week until stable titer levels were obtained. At each bleeding, the serum antibody titer was measured against the biotinylated peptide, GIATKGLNGP-k(Biotin) (SEQ ID NO: 5) coated on streptavidin coated microplates (Roche Diagnostics, Germany). The mice with the highest antibody titer and best reactivity towards the selection peptide, GIATKGLNGP (SEQ ID NO: 1), was chosen for fusion (data not shown). The mouse selected was boosted intraperitoneally (i.p.) with 100 μg KLH-conjugate in 100 μL 0.9% sodium chloride solution three days before sacrificed for fusion. The fusion and antibody screening process was performed using standard techniques. Briefly, the spleen was surgically removed for isolation of splenocytes which was fused with murine myeloma cells, SP2/0-Ag14 (ATCC®CRL-1581™) Hybridoma cells were selected by using HAT (hypoxanthine Aminopterin Thymidine) medium. Supernatants were screened by an indirect ELISA, where the biotinylated peptide, GIATKGLNGP-k(Biotin) (SEQ ID NO: 5) was coated on streptavidin-precoated microplates. Standard limiting dilution process was carried out to select single cells. The isotype of antibody in supernatant was tested using the isotype determination kit, SBA Clonotyping System-HRP (5300-05, Southern Biotech). Single-cell derived hybridoma were transferred to 24-well plate to allow them for further growth, and eventually scale up from T25 to T175 flasks. The supernatants were collected and filtered with 0.2 μm filter before applying to a 1 mL HiTrap Protein G HP column (cat #17-0404-01, GE healthcare) for antibody purification.

### ⁴⁷⁹GIATKG Specific Immuno-Assay (Col10neo)

A purified monoclonal antibody was first tested for peptide-binding specificity by three synthetic peptides, selection peptide (GIATKGLNGP; SEQ ID NO: 1), elongation of the selection peptide (AGIATKGLNGP; SEQ ID NO: 3) and truncation of the selection peptide (IATKGLNGP; SEQ ID NO: 4), and then used in a competitive immunoassay. After optimization with buffer, temperature, incubation time and concentrations for key reagents, the final protocol for detection of ⁴⁷⁹GIATKG (SEQ ID NO: 2) assay was developed as follows: a 96-well streptavidin pre-coated micro plate was coated with 1 ng/ml biotinylated peptide, GIATKGLNGP-k(Biotin) (SEQ ID NO: 5) dissolved in 50 mM PBS-BTB buffer (phosphate-buffered saline with bovine serum albumin and Tween-20, pH7.4) for 30 min at 20° C. Then, the plate was washed 5 times by standard wash buffer (20 mM Tris, 50 mM NaCl, pH 7.2). 20 μL of the peptide standards, kit controls or samples were added to appropriate wells, followed by 100 μL of 23 ng/ml monoclonal antibody in 50 mM PBS-BTB buffer containing 5% Osteocalcin EIA Puf-Liq (Roche diagnostics, Germany), and incubated overnight (20±1 hr) at 4° C. After 5 times wash, 100 μL of goat anti-mouse secondary antibody (115-035-003, Jackson ImmunoResearch) was added and incubated for 1 hr at 20° C. After this step, the plate was washed for 5 times with wash buffer. Finally, 100 μL 3,3′,5,5′-tetramethylbenzidine (TMB) was added to each well and incubated for 15 min at 20° C. in the dark. The colorimetric reaction was stopped by adding 100 μL stopping solution (1% H₂SO₄) and measured at 450 nm with 650 nm as the reference.

The inter- and intra-plate variation were determined by 10 independent runs of quality control panel (three human sera and two peptide standard) in duplicate. The lower limit of detection (LLOD) was calculated as 3SD of the mean value of 21 zero standard. Multiple human serum or plasma samples were assayed neat, 2, 4, 6, 8, 16-fold in incubation buffer. Percent recovery was calculated as the measured concentration divided by the expected concentration corrected for dilution.

### In Vitro Cleavage of Human Cartilage

Articular cartilage biopsies from osteoarthritis (OA) patients who underwent knee replacement surgery were obtained from Gentofte Hospital (Gentofte, Denmark). The retrieval of the specimens complied with international ethical guidelines for handling human samples and patient information. All participants signed an informed consent and the study was approved by Danish authority. To identify the enzymes responsible for the cleavage of the A⁴⁷⁸-⁴⁷⁹G bond, cartilage was cleaved by numerous proteases including matrix metalloproteases (MMP2, MMP9 and MMP13), Cathepsins (CatK, CatB and CatS), and A disintegrin and metalloprotease domains with thrombospondins motifs (ADAMTS-4 and ADAMTS-5). 30 mg of snap-frozen and crushed cartilage with 1 μg each enzyme were placed in a 0.5 ml Eppendorf with 250 μL digestion buffer (digestion buffer for MMPs: 100 mM Tris, 100 mM NaCl, 10 mM CaCl₂ and 2 mM zinc acetate, pH 8.0; digestion buffer for ADAMTSs: 50 mM Tris, 100 mM NaCl and 10 mM CaCL₂, pH 7.5; digestion buffer for Cathepsins: 25 mM NA₂HPO₄, 150 mM NaCl, 2 mM EDTA, 2 mM DTT, pH 6.5). The digestion was carried out on one day in 2 replicates. 5 mM EDTA (broad-spectrum inhibitor for MMPs and ADAMTSs) and 5 mM E64 (broad-spectrum inhibitor for Cathepsins) were added to stop the reaction. All supernatants were stored at −80° C. prior to use.

### Immunohistochemistry (IHC)

Cartilage with bone specimens for IHC were from the study in collaboration with Frederikshavn Hospital (Denmark) approved by Danish authority (N-20110031). All participants signed an informed consent. The specimens isolated from OA patients who underwent knee replacement surgery were fixed and decalcified, and then embedded in paraffin. 5 μm-thick cartilage sections were melt at 60° C. for 1 hr and deparrifined and hydrated, followed by antigen retrieval using Pronase E (cat #10165921001, Roche) at 37° C. for 10 min. The sections were treated by 0.5% casein in Tris Buffered Saline (TBS) for 30 min at room temperature to block unspecific binding. Immunostaining was performed by antibody NB117-2F4 developed in this study and previously developed antibody NB509-11G8. Immunoreactivity was visualized with peroxidase labeled anti-Mouse and diaminobenzidine (DAB, Dako, Denmark). Counterstaining was with Mayer's hematoxylin. The pictures were taken using an Olympus microscope BX60 equipped with an Olympus C5050 digital camera.

### Study Participants

Plasma samples were retrieved from two studies, the C4Pain study approved by local ethics committee (N-20100094) [24] and the NYUHJD Progression study approved by local ethics committee [25, 26]. All participants provided informed consent prior to enrollment.

Briefly, the C4Pain is a cross-sectional study. It comprised 281 individuals with no joint degeneration to severe joint degeneration using the Kellgren/Lawrance (K/L) grading scale (K/L 0-4). The plasma samples from 253 enrollees were measured in the present study due to insufficient volume of other 28 samples.

The NYUHJD Progression study comprised 21 non-OA healthy controls (K/L≤1 and no pain in either knee), 146 OA patients (K/L≥2) and 36 rheumatoid arthritis (RA) patients at baseline. 146 OA patients were further followed up for 24 months. The radiographic assessments were taken at baseline and 24 months and plasma were collected at baseline. Plasma samples from 20 non-OA healthy controls, 142 OA subjects and 34 RA subjects at baseline were available for measurement in the present study. However in the present study, we only investigated the level of Col10neo in different groups at baseline. Future studies will continue to move toward a more complete picture of Col10neo as a potential prognostic biomarker of radiographic progression in OA.

### Statistics

Data were analyzed using GraphPad Prism 6 or MedCalc 16.8. For normality check, a Shaprio-Wilk test for all variables of interest was performed. Between group comparison for age, BMI and VAS score, the one-way ANOVA with post-hoc Tukey-Kramer test was used. Plasma Col10neo data were logarithmic transformed in all analyses. P value<0.05 was considered statistically significant. One asterisk (*) if p<0.05; two (**) if p<0.01; three (***) if p<0.001 and four (****) if p<0.0001.

### Results

### Peptide Selection

Sequence alignment of the selected peptide across species 2 fragments sharing the same free C-terminal end, ⁴⁶³PGSKGDPGSPGPPGPA⁴⁷⁸ (SEQ ID NO: 7) and ⁴⁶⁵SKGDPGSPGPPGPA⁴⁷⁸ (SEQ ID NO: 8) were identified by mass spectrometry in urine of OA patients, indicating the presence of a cleavage site existing between A⁴⁷⁸-⁴⁷⁹G. A 10aa peptide from the free N-terminus generated by the cleavage, ⁴⁷⁹GIATKGLNGP (SEQ ID NO: 1), was chosen for immunization. The blast shows that among human proteins the sequence is unique to type X collagen alpha-1. Sequence similarity across species shows 100% identity between human and mouse, while a mismatched aa is contained in rat or bovine compared to human sequence.

TABLE 1
The sequence alignment of the chosen peptide across
species

| Species | Sequence | Gene name | Acession | DataBase |
|---|---|---|---|---|
| Homo sapiens¹ | GIATKGLNGP | COL10A1 | Q03692 | UniProt |
| Rattus | GIVTKGLNGP | COL10A1 | A0A0G2K7A5 | UniProt |
| norvegicus² | | | | |
| Mus Musculus³ | GIATKGLNGP | COL10A1 | Q05306 | UniProt |
| Bos taurus⁴ | GIAVKGLNGP | COL10A1 | P23206 | UniProt |
| Canis lupus | GVATKGLNGP | COL10A1 | Q2HNR1 | UniProt |
| familiaris⁵ | | | | |

¹Human (SEQ ID NO: 1); ²Rat (SEQ ID NO: 9); ³Mouse (SEQ ID NO: 10); ⁴Bovine (SEQ ID NO: 11); ⁵Dog (SEQ ID NO: 12)

### Technical Performance of the Col10neo Assay

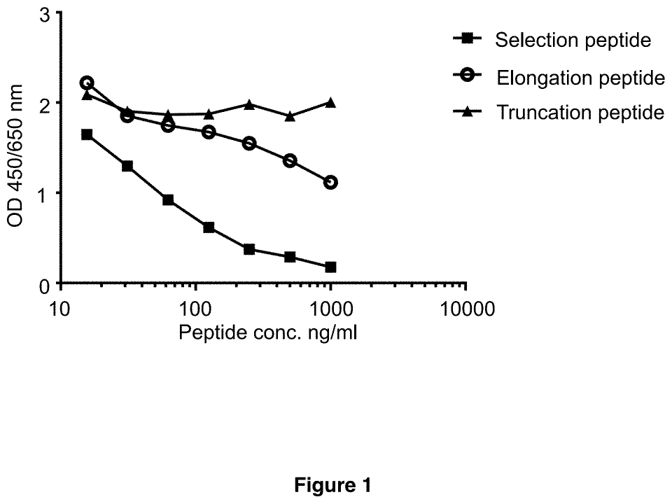

The monoclonal antibody 2F4 (isotype: IgG2b, K) targeting ⁴⁷⁹GIATKG (SEQ ID NO: 2) was produced from hybridoma and purified by HiTrap Protein G affinity column (Cat #17-0404-01, GE Healthcare). In test for peptide-binding specificity, the reactivity of 2F4 toward a biotinylated synthetic peptide, GIATKGLNGP-k(Biotin) (SEQ ID NO: 5), was completely displaced by adding of 1000 ng/mL selection peptide. In contrast, a slight or no displacement was observed with elongation of the selection peptide or truncation of the selection peptide at the same concentration (FIG. 1). This indicated the developed antibody 2F4 was specific for the selection peptide.

The antibody 2F4 showing great specificity was therefore applied in a competitive ELISA assay, Col10neo. The technical performance of this assay is summarized and listed in table 2. The IC50 was 41.9 ng/mL. The intra-assay coefficient variation (CV %) was 3% and the inter-assay CV % was 11.8. The measurement range was 8-250 ng/mL. The linearity was good over a wide range of 4- to 32-fold dilution of serum and 8- to 64-fold dilution of EDTA-anticoagulated plasma.

TABLE 2
Summary of technical performance of Col10neo assay

| Assay specifications | Col10neo |
|---|---|
| IC50, ng/mL | 41.9 |
| Intra-assay, CV % | 3 |
| Inter-assay, CV % | 11.9 |
| Lower limit of detection , ng/mL | 0.127 |
| Measurement range, ng/mL | 8-250 |

### Cathepsin K-Derived ⁴⁷⁹GIATKG

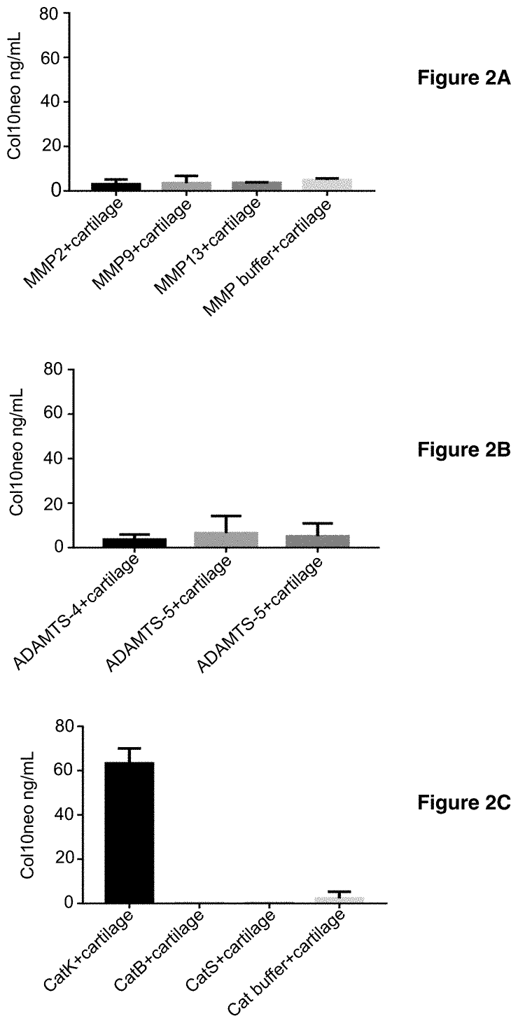

To investigate the responsible enzyme for cleaving A⁴⁷⁸-⁴⁷⁹G bond, several proteases were individually incubated with human cartilage. All proteases were at the same enzyme concentration and all incubations were performed for the same incubation time. The relative efficiency for each protease to generate the neo-epitope of ⁴⁷⁹GIATKGLNGP (SEQ ID NO: 1) was evaluated by applying to the Col10neo assay. There was no increased level of Col10neo in any of the tested MMPs or ADAMTSs solution compared to the one without adding proteases in the digestion buffer (FIGS. 2A and 2B). Conversely, Cathepsin K yielded the largest amount indicating its ability of releasing the fragment carrying the neo-epitope of ⁴⁷⁹GIATKGLNGP (SEQ ID NO: 1) (FIG. 2C).

### Immunolocalization of ⁴⁷⁹GIATKG in Cartilage

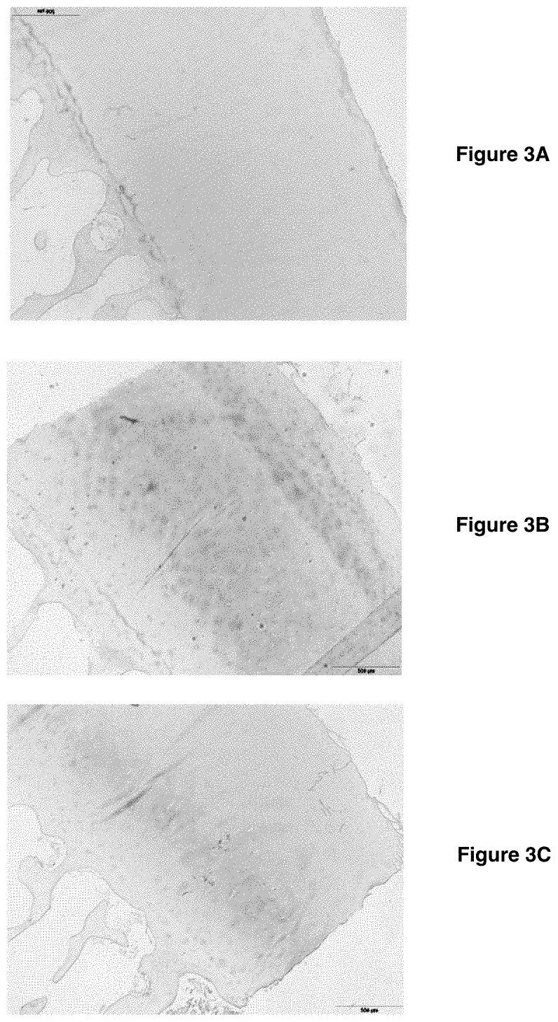

To further define the generation and distribution of ⁴⁷⁹GIATKG (SEQ ID NO: 2), consecutive sections of articular cartilage from a TKR (total knee replacement) patient were stained with anti-⁴⁷⁹GIATKG (SEQ ID NO: 2) (2F4), normal mouse IgG (negative control) and anti-C terminus of type X collagen (11G8-disclosed in WO 2014/180992) as well. We did not observe any staining with normal mouse IgG (FIG. 3A). In line with previous studies, it was found that type X collagen detected by 11G8 occurred in the extracellular matrix of chondrocytes in the deep zone, but it was absent from the region of calcified cartilage (FIG. 3B). Surprisingly, the intense staining of ⁴⁷⁹GIATKG (SEQ ID NO: 2) was seen in the extracellular matrix of chondrocytes from all zones in cartilage (FIG. 3C). The staining of neo-epitope demonstrated that type X collagen released into the extracellular matrix had undergone further proteolytic processing.

### Association Between K/L Grade and Plasma Col10neo Levels in the C4Pain Study

The 253 participants from the C4Pain study were categorized into 4 groups based on the K/L grade. The demographic characteristics of these 4 groups are summarized in table 3.

TABLE 3
Demographics of subjects in the C4Pain study

| K/L | No. of | No. Of | | VAS | Age, | BMI, |
|---|---|---|---|---|---|---|
| grade | men | women | Total | grade | years | kg/m² |
| 0-1 | 23 | 27 | 50 | 37 ± 30 | 61.8 ± 8.5 | 26.4 ± 3.1 |
| 2 | 79 | 66 | 145 | 42 ± 29 | 64.6 ± 7.3 | 28.2 ± 3.8 |
| 3 | 17 | 19 | 36 | 56 ± 21 | 64.3 ± 7.1 | 29.3 ± 5.6 |
| 4 | 12 | 10 | 22 | 54 ± 24 | 67.8 ± 7.7^{$} | 29.5 ± 3.9 |

Except where indicated otherwise, values are the mean±SD.
Vas grade=maximal pain intensity for the last
24 hour, BMI=body mass index.
$, P<0.05 compare to K/L0-1 group

As the mean age of the participants was >60 years, the few participants with a K/L 0 or K/L of 1 were classified into the same group. 57% of the subjects involved in this study were with a K/L of 2 and assigned to group 2. There was no significant difference in gender distribution within each group. The mean age of K/L4 was significantly higher than K/L 0-1 group (p<0.005). There was a clear but not significant trend of increased BMI with an increase in K/L grade. The mean value of VAS score, defined as the maximal pain intensity for the last 24 hours, was not significantly different from each group.

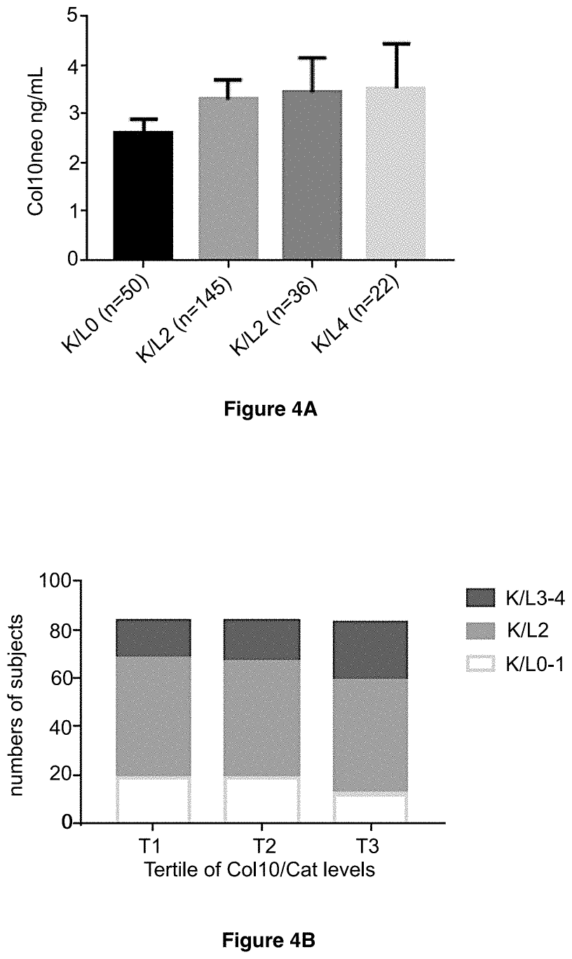

The mean±95CI % concentrations of Col10neo for participants in 4 groups from KL 0-4 were 2.6 [2.316-2.884] μg/mL, 3.288[2.885-3.691] μg/mL, 3.435[2.729-4.141] μg/mL and 3.517[2.599-4.435] μg/mL, respectively. There was a trend toward increased Col10neo levels with a greater K/L grade, but that did not reach statistical significance (FIG. 4A). The subjects were divided into tertiles based on the Col10neo levels and the distribution of K/L grade was compared (FIG. 4B). The number of subjects with a K/L 3-4 was greatest in those in the highest tertile of Col10neo.

### Plasma Col10neo Levels in the NYUHJD Progression Study

The result from the C4Pain study caused us to investigate the potential of using Col10neo as a diagnostic biomarker in the NYUHJD Progression study which consists of non-OA healthy control, OA and RA. The percentage of female participants was higher in OA and RA groups than in the healthy control group. The mean age was significantly different in control and OA groups. However, the subjects were significantly younger in the RA group than in the OA group, since RA can occur at any age but has its peak between ages 30-55. There was a slight difference in BMI between healthy control and OA.

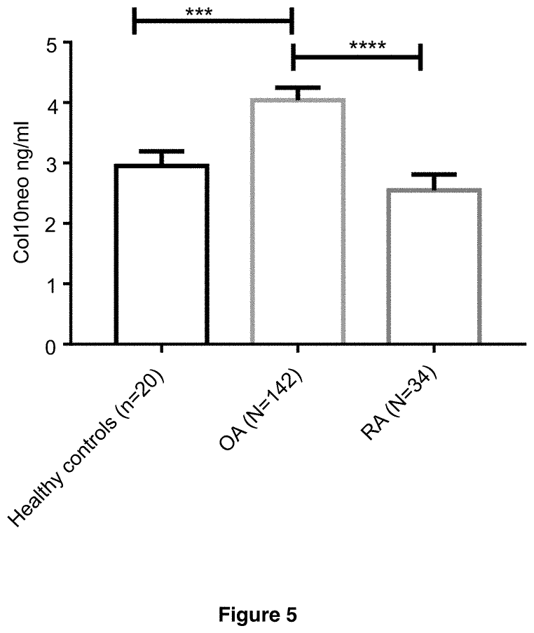

The plasma Col10neo (mean±95CI %) in control, OA and RA was 2.953[2.711-3.194]μg/mL, 4.04[3.835-4.246]μg/mL and 2.548[2.285-2.81]μg/mL, respectively (FIG. 5). Plasma Col10neo was statistically higher in OA than healthy control (p=0.0002) or RA (p<0.0001). No significant difference between the healthy control and RA was found. When the data was adjusted for age and gender, the level of Col10neo remained significantly increased in OA with respect to the control (p=0.003) or RA (p<0.0001) (Table 5).

Surprisingly, when adjusted for age and gender the difference between the control and Col10neo became significant for RA (p=0.0084; Table 5).

TABLE 5
ANCOVA analysis of plasma Col10neo levels adjusted
for age and gender.

| Factors | | Mean difference | Std. Error | P ^{a} | 95% CI a |
|---|---|---|---|---|---|
| OA | - healthy | 0.1156 | 0.02913 | 0.0003 | 0.04527 |
| | control | | | | to |
| | | | | | 0.1860 |
| | - RA | 0.2200 | 0.02425 | <0.0001 | 0.1614 |
| | | | | | to |
| | | | | | 0.2786 |
| Healthy | - OA | −0.1156 | 0.02913 | 0.0003 | −0.1860 |
| control | | | | | to- |
| | | | | | 0.04527 |
| | - RA | 0.1044 | 0.03446 | 0.0084 | −0.1860 |
| | | | | | to |
| | | | | | −0.04527 |

^{a} Bonferroni corrected

### CONCLUDING REMARKS

It has been demonstrated that the herein disclosed col10neo assay is useful in the evaluation of OA.

It is herein proposed that this usefulness may extend to evaluating other diseases associated with collagen type X alpha-1, such as ankylosing spondylitis.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### REFERENCES

1. Abhishek, A. and M. Doherty, Pathophysiology of articular chondrocalcinosis role of ANKH. Nat Rev Rheumatol, 2011; 7:96-104.
2. Kronenberg, H. M., Developmental regulation of the growth plate. Nature, 2003; 423:332-6.
3. Pfander, D., B. Swoboda, and T. Kirsch, Expression of early and late differentiation markers (proliferating cell nuclear antigen, syndecan-3, annexin VI, and alkaline phosphatase) by human osteoarthritic chondrocytes. Am J Pathol, 2001; 159:1777-83.
4. von der Mark, K., et al., Type X collagen synthesis in human osteoarthritic cartilage. Indication of chondrocyte hypertrophy. Arthritis Rheum, 1992; 35:806-11.
5. Fuerst, M., et al., Calcification of articular cartilage in human osteoarthritis. Arthritis Rheum, 2009; 60:2694-703.
6. Dreier, R., Hypertrophic differentiation of chondrocytes in osteoarthritis: the developmental aspect of degenerative joint disorders. Arthritis Res Ther, 2010; 12:216.
7. Pitsillides, A. A. and F. Beier, Cartilage biology in osteoarthritis—lessons from developmental biology. Nat Rev Rheumatol, 2011; 7:654-63.
8. van der Kraan, P. M. and W. B. van den Berg, Chondrocyte hypertrophy and osteoarthritis: role in initiation and progression of cartilage degeneration? Osteoarthritis Cartilage, 2012; 20:223-32.
9 Frischholz, S., et al., J. Biol. Chem., 1998; 273:4547.
10. Yamaguchi, N., et al. J. Biol. Chem., 1989; 264:16022.
11. Olsen, B. J., and Ninomiya, Y., in: “Guidebook to the Extracellular Matrix and Adhesion Proteins”, Kreis, T., and Vale, R. (eds.), Oxford University Press, Oxford, pp. 32-48 (1993).
12. Schmid, T. M., and Linsenmayer, T. F., in: “Structure and Function of Collagen Types”, Mayne, R., and Burgeson, R. E. (eds.), Academic Press Inc., pp. 223-259 (1987).
13. Rucklidge, G. J., et al., Matrix Biol., 1996; 15; 73.
14. Aigner, T., et al., Histochem. Cell Biol., 1997; 107; 435.
15. Girkontaite, I., et al., Matrix Biol., 1996; 15; 231.
16. Gerstenfeld, L. C. and F. D. Shapiro, Expression of bone-specific genes by hypertrophic chondrocytes: implication of the complex functions of the hypertrophic chondrocyte during endochondral bone development. J Cell Biochem, 1996; 62:1-9.
17. Wei, F., et al., Activation of Indian hedgehog promotes chondrocyte hypertrophy and upregulation of MMP-13 in human osteoarthritic cartilage. Osteoarthritis Cartilage, 2012; 20:755-63.
18. Dong, Y. F., et al., Wnt induction of chondrocyte hypertrophy through the Runx2 transcription factor. J Cell Physiol, 2006; 208:77-86.
19. Horner, A., et al., Immunolocalisation of vascular endothelial growth factor (VEGF) in human neonatal growth plate cartilage. J Anat, 1999; 194:519-24.
20. Tsuchiya, A., et al., Expression of mouse HtrA1 serine protease in normal bone and cartilage and its upregulation in joint cartilage damaged by experimental arthritis. Bone, 2005; 37:323-36.
21. Huebner, J. L., et al., Transglutaminase 2 is a marker of chondrocyte hypertrophy and osteoarthritis severity in the Hartley guinea pig model of knee OA. Osteoarthritis Cartilage, 2009; 17:1056-64.
22. Fitzgerald, J. B., et al., Shear- and compression-induced chondrocyte transcription requires MAPK activation in cartilage explants. J Biol Chem, 2008; 283:6735-43.
23. Goldring, M. B., et al., Defining the roles of inflammatory and anabolic cytokines in cartilage metabolism. Ann Rheum Dis, 2008; 67:75-82.
24. Arendt-Nielsen L, Eskehave T N, Egsgaard L L, et al. Association between experimental pain biomarkers and serologic markers in patients with different degrees of painful knee osteoarthritis. Arthritis Rheumatol (Hoboken, N.J.). 2014; 66(12):3317-3326. doi:10.1002/art.38856.
25. Attur M, Statnikov A, Samuels J, et al. Plasma levels of interleukin-1 receptor antagonist (IL1Ra) predict radiographic progression of symptomatic knee osteoarthritis. Osteoarthr Cartil. 2015; 23(11):1915-1924. doi:10.1016/j.joca.2015.08.006.
26. Attur M, Krasnokutsky S, Statnikov A, et al. Low-Grade Inflammation in Symptomatic Knee Osteoarthritis: Prognostic Value of Inflammatory Plasma Lipids and Peripheral Blood Leukocyte Biomarkers. Arthritis Rheumatol. 2015; 67(11):2905-2915. doi:10.1002/art.39279.

1: An antibody, wherein said antibody specifically reacts with an N-terminus neo-epitope of collagen type X alpha 1 comprised in the amino acid sequence H₂N-GIATKGLNGP (SEQ ID NO: 1).

2: The antibody as claimed in claim 1, wherein said antibody specifically binds to the N-terminus amino acid sequence H₂N-GIATKG (SEQ ID NO: 2).

3: The antibody as claimed in claim 1, wherein the antibody does not specifically recognise or bind an N-extended elongated version of said N-terminus amino acid sequence or an N-truncated shortened version of said N-terminus amino acid sequence.

4: The antibody as claimed in claim 1, wherein said antibody is a monoclonal antibody or fragment thereof.

5: A cell line producing the monoclonal antibody as claimed in claim 4.

6: A method of immunoassay for detecting in a biological sample fragments of collagen type X alpha 1 comprising an N-terminus neo-epitope amino acid sequence H₂N-GIATKGLNGP (SEQ ID NO: 1), said method comprising contacting said biological sample comprising said N-terminus neo-epitope amino acid sequence H₂N-GIATKGLNGP (SEQ ID NO: 1) with an antibody as claimed in claim 1, and determining the amount of binding of said antibody.

7: The method of immunoassay as claimed in claim 6, wherein the detection is quantitative.

8: The method of immunoassay as claimed in claim 6, wherein said method is used to detect and/or quantify the amount of fragments of collagen type X alpha 1 comprising the N-terminus neo-epitope amino acid sequence H₂N-GIATKGLNGP (SEQ ID NO: 1) in a biofluid.

9: The method of immunoassay as claimed in claim 8, wherein said biofluid is a patient derived biofluid.

10: The method of immunoassay as claimed in claim 8, wherein said biofluid is blood, urine, synovial fluid, serum or plasma

11: The method of immunoassay as claimed in claim 6, wherein said method is a competition assay or a sandwich assay.

12: The method of immunoassay as claimed in claim 6, wherein said method is a radioimmunoassay or an enzyme-linked immunosorbent assay.

13: The method of immunoassay as claimed in claim 6, wherein said method further comprises correlating the quantity of fragments of collagen type X alpha 1 comprising said N-terminus neo-epitope amino acid sequence H₂N-GIATKGLNGP (SEQ ID NO: 1) determined by said method with standard disease samples of known disease severity to evaluate the severity of a disease associated with collagen type X alpha 1.

14: The method of immunoassay as claimed in claim 6, wherein said method further comprises comparing the quantity of said fragments of collagen type X alpha 1 comprising said N-terminus neo-epitope amino acid sequence H₂N-GIATKGLNGP (SEQ ID NO: 1) determined by said method with standard values associated with healthy subjects to evaluate the presence and/or severity of a disease associated with collagen type X alpha 1.

15: The method of immunoassay as claimed in claim 13, wherein the disease associated with collagen type X alpha 1 is osteoarthritis.

16: An assay kit for determining the quantity of fragments of collagen type X alpha 1 comprising the N-terminus neo-epitope amino acid sequence H₂N-GIATKGLNGP (SEQ ID NO: 1) in a biological sample, said kit comprising an antibody as claimed in claim 1 and at least one of:
a streptavidin coated 96 well plate
a biotinylated peptide H₂N-GIATKGLNGP-L-Biotin (SEQ ID NO: 5), wherein L is an optional linker
a secondary antibody for use in a sandwich immunoassay
a calibrator peptide comprising the sequence H₂N-GIATKGLNGP (SEQ ID NO: 1)
an antibody biotinylation kit
an antibody HRP labeling kit
an antibody radiolabeling kit
an assay visualization kit.
